(19) United States
(12) Patent Application Publication
(10) Pub. No.: US 2007/0173837 A1
(43) Pub. Date: Jul. 26, 2007

(54) BONE FIXATION AND DYNAMIZATION DEVICES AND METHODS

(75) Inventors: Elaine Chan, Sugar Land, TX (US); Peter Yang, Silver Spring, MD (US); Alex Gordon, Kenilworth, IL (US); Eric Vu, Sugar Land, TX (US); Cynthia Chang, Auburn, AL (US); Michael Liebschner, Pearland, TX (US)

Correspondence Address: CONLEY ROSE, P.C.;David A. Rose, P. O. BOX 3267, HOUSTON, TX, 77253-3267, US

(73) Assignee: William Marsh Rice University, Houston, TX (US)

(21) Appl. No.: 11/560,770

(22) Filed: Nov. 16, 2006

Related U.S. Application Data
(60) Provisional application No. 60/738,381, filed on Nov. 18, 2005.
(60) Provisional application No. 60/744,306, filed on Apr. 5, 2006.

(51) Int. Cl. A61F 2/30

(52) U.S. Cl. 606/63

(57) ABSTRACT

A bone fixation and dynamization device comprising a first member having a first end and a second end; a second member having a first end and a second end, wherein the first end of the second member is coupled to the second end of the first member body, wherein the first member is linearly moveable relative to the second member; an actuator coupled to the first member; a feedback controller coupled to the actuator; an elongate rod having an actuator end coupled to the actuator and a fixed end fixed to the second member, wherein the actuator is operable to move the rod and the second member linearly relative to the first member responsive to the feedback controller; at least one bone engagement pin extending from the first member; and at least one bone engagement pin extending from the second member.

### CROSS-REFERENCE TO RELATED APPLICATIONS

This application claims benefit of U.S. provisional application Ser. No. 60/738,381 filed Nov. 18, 2005, and entitled “Bone Fixation Device,” which is hereby incorporated herein by reference in its entirety. This application also claims benefit of U.S. provisional application Ser. No. 60/744,306 filed Apr. 5, 2006, and entitled “Bone Fixation Device,” which is hereby incorporated herein by reference in its entirety.

### STATEMENT REGARDING FEDERALLY SPONSORED RESEARCH OR DEVELOPMENT

Not applicable.

### BACKGROUND

1. Field of the Invention

The invention relates generally to devices and methods to stabilize a bone fracture and promote healing of the fracture. More particularly, the present invention relates to devices and methods to promote healing of a bone fracture by actively inducing micromovement of the fractured bone segments at the bone fracture site.

2. Background of the Invention

Over 25 million people in the United States will experience some musculoskeletal injury each year at a total cost of over $250 billion. Among the most common musculoskeletal injuries are broken bones. Musculoskeletal injuries, including bone fractures, may be caused by numerous factors. For example, motor vehicle accidents, falls, direct impacts to joints or bones, the application of repetitive forces (e.g., such as may result from running) may cause various musculoskeletal injuries. It is estimated that over 1.5 million insufficiency fractures each year are caused during normal daily activities and are related to senile osteoporosis and primary osteoporosis.

In general, a bone will likely fracture if more pressure or force is placed on the bone than the bone can stand. Thus, two factors in determining whether a bone fracture may occur are (1) the pressure or force placed on the bone by the event, and (2) the strength of the bone (i.e., how much pressure or force the bone can withstand without breaking). Therefore, risks for a bone fracture increase as a bone weakens. Bones may weaken for a variety of reasons including aging, disease, osteoporosis, bone loss, etc. Weakening of bones is of particular concern in low gravity and microgravity environments (e.g., astronauts in low-earth orbit or outer space) that tend to induce bone loss, as well as with bed ridden and paraplegic patients who are unable to load their musculoskeletal system.

When a bone is fractured, the two or more bone fragments are re-aligned and stabilized so that the fragments can properly heal together. The bone fragments may be aligned and stabilized with an internal bone fixation device and/or with an external bone fixation device. An internal fixation device is typically a plate that is surgically attached to the bone across the fracture site by screws or pins, or a rod that is placed inside the medullary canal of long bones and held in place by screws. While an external bone fixation device is external to the body and may be attached to the bone percutaneously (i.e., through the skin and intervening tissue) by screws or pins, or non-invasively coupled to the bone via a cast. In either case, internal or external, the devices are intended to align and stabilize the bone during the healing process.

For complicated fractures, external fixation followed by dynamization is often employed. In general, dynamization refers to the micromovement (e.g., movements of 1 mm or less) of the fractured bone segments at the fracture site. Dynamization results in the partial loading of the fractured bone, which has been shown to promote and stimulate bone healing, and potentially increase bone healing rates. For example, studies have shown that partial loading of a fractured bone via micromovement on the scale of 1 mm at 0.5 Hz increases the rate of bone healing. It is believed that dynamization stimulates the proliferation of the periosteal callus in the early phase and accelerates the remodeling and hypertrophic response of normal bone cells late in the healing phase. It is also hypothesized that low-magnitude, higher frequency mechanical stimuli simulate the small vibrations applied to bones by flexing muscles under normal conditions. These 10-100 Hz frequencies may also induce a signal for bone formation. An increase in micromovement has also been shown to increase blood flow to the fracture area by up to 25%. The increased vascular response may also play a significant role in organizing new bone formation.

Most conventional dynamization techniques rely on the normal physical motion and load bearing activities of the patient which transmit forces and micromovements to the fractured bone segments at the fracture site. However, for patients who are unable or unwilling to load their bones through normal physical activities (e.g., bedridden, elderly, traumatized, or paraplegic patients), such conventional dynamization techniques may not be sufficient to achieve increased bone healing rates. In addition, such conventional dynamization techniques may not be effective to enhance healing rates in fracture bones that bear minimal or no loads during the normal physical activities of the patient. Further, in low gravity or microgravity environments, normal physical activities may not result in sufficient loading of the fractured bone segments necessary to enhance bone fracture healing. Low gravity environments include environments in which the gravitational acceleration and resulting gravitational force is less than that at the earth's surface (e.g., in low-earth orbit or in outer space). In such an environment, the loads and forces transmitted to a fractured bone by normal physical activities and motion are greatly reduced due to the reduction in gravity. In some cases (e.g., zero gravity), patient movement and physical activity results in effectively zero external loading of bones.

Accordingly, there remains a need in the art for devices and methods that can align a fractured bone, stabilize the fractured bone, promote healing, and/or accelerate healing of the fractured bone. Such devices and methods would be well received if they offered the potential to enhance the healing of fractured bones that do not bear sufficient loads during normal physical activities, for patients who are unable or unwilling to physically load their bones, and promote bone healing in low gravity or microgravity environments.

### BRIEF SUMMARY OF SOME OF THE PREFERRED EMBODIMENTS

Disclosed herein is a bone fixation and dynamization device comprising a first member having a first end and a second end; a second member having a first end and a second end, wherein the first end of the second member is coupled to the second end of the first member body, wherein the first member is linearly moveable relative to the second member; an actuator coupled to the first member; a feedback controller coupled to the actuator; an elongate rod having an actuator end coupled to the actuator and a fixed end fixed to the second member, wherein the actuator is operable to move the rod and the second member linearly relative to the first member responsive to the feedback controller; at least one bone engagement pin extending from the first member; and at least one bone engagement pin extending from the second member.

Further disclosed herein is a method for fixing and dynamizing a fracture in a bone, comprising (b) providing a bone fixation and dynamization device, wherein the bone fixation and dynamization device comprises a first member; a second member coupled to the first member, wherein the second member is operable to move linearly relative to the first member; an actuator coupled to the first member; a feedback controller coupled to the actuator; and an elongate rod having an actuator end coupled to the actuator and a fixed end fixed to the second member, wherein the actuator is operable to move the second member linearly relative to the first member responsive to the feedback controller; (b) connecting the first member to a first bone segment on one side of the fracture; (c) connecting the second member to a second bone segment on the other opposite side of the fracture; and (d) applying oscillating micromovements to the first and second bone segments with the bone fixation and dynamization device.

Further disclosed herein is a method of dynamizing a fracture in a bone having a longitudinal axis comprising engaging a bone segment on each side of the fracture with at least one bone engagement pin; oscillating the bone engagement pins on either side of the fracture linearly relative to one another; applying linear oscillating micromovements the bone segments on either side of the fracture; and controlling the micromovements via feedback control.

Thus, embodiments described herein comprise a combination of features and advantages intended to address various shortcomings associated with certain prior devices. The various characteristics described above, as well as other features, will be readily apparent to those skilled in the art upon reading the following detailed description of the preferred embodiments, and by referring to the accompanying drawings.

### BRIEF DESCRIPTION OF THE DRAWINGS

For a detailed description of the preferred embodiments of the invention, reference will now be made to the accompanying drawings in which:

FIG. 1 is a perspective view of an embodiment of a bone fixation and dynamization device;

FIG. 2 is a side view of the bone fixation and dynamization device of FIG. 1;

FIG. 3 is a bottom view of the bone fixation and dynamization device of FIG. 1;

FIG. 4 is an enlarged schematic view of an embodiment of the coupling between the actuator and connecting rod of FIG. 1;

FIG. 5 is a perspective view of another embodiment of a bone fixation and dynamization device;

FIG. 6 is a side view of the bone fixation and dynamization device of FIG. 5;

FIG. 7 is a partial side schematic view of the bone fixation and dynamization device of FIG. 1 percutaneously coupled to a fractured bone; and

FIG. 8 is an enlarged schematic view of the bone fracture site of FIG. 4;

### DETAILED DESCRIPTION OF THE PREFERRED EMBODIMENTS

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

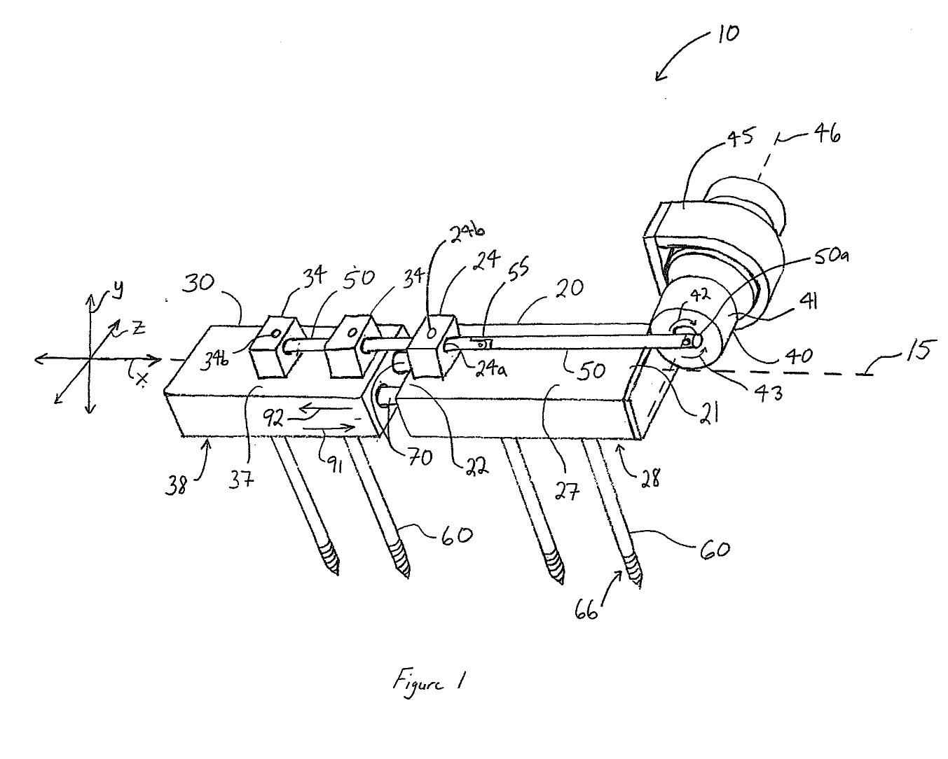

For purposes of this discussion, orthogonal x-, y-, and z-axes are shown in several Figures (e.g., FIGS. 1-3 and 5-8) to aid in understanding the descriptions that follow. In general, the x-axis defines longitudinal positions and movement, the y-axis defines vertical positions and movement, and the z-axis defines lateral positions and movement. The set of coordinate axes (x-, y-, and z-axes) are consistently maintained throughout although different views (e.g., front view, side view, etc.) may be presented.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection, or through an indirect connection via other devices and connections.

### Bone Fixation and Dynamization Devices

Referring now to FIGS. 1-3, an embodiment of a bone fixation and dynamization device 10 is illustrated. Bone fixation and dynamization device 10 comprises a first member 20, a second member 30, an actuator 40, pins 60, and a connecting rod 50. First member 20 includes a first end 21, a second end 22, an upper surface 27, and a lower surface 28. Likewise, second member 30 includes a first end 31, a second end 32, an upper surface 37, and a lower surface 38. As will be explained in more detail below, bone fixation and dynamization device 10 may be employed to stabilize a fractured bone, immobilize a fractured bone, lengthen a bone, provide active dynamization (e.g., oscillating micromovements) to a fractured bone, or combinations thereof.

First member 20 is linearly coupled to second member 30. Specifically, second end 22 of first member 20 is linearly coupled to first end 31 of second member 30 by a pair of parallel guide shafts 70. As used herein, the terms “linear” and “linearly” may be used to refer to positions and/or connections generally extending or arranged in a line or along a line. For instance, in the embodiment shown in FIG. 1, first member 20 and second member 30 are connected end-to-end and generally share the same longitudinal axis 15. Thus, first member 20 and second member 30 may be described as being linearly coupled to each other.

Each guide shaft 70 has a first member end 71 at least partially disposed in a mating shaft bore 26 in second end 22 of first member 20, and a second member end 72 at least partially disposed in a mating shaft bore 36 in first end 31 of second member 30. First member end 71 and/or second member end 72 of each guide shaft 70 slidingly engages bore 26 and/or bore 36, respectively. Thus, guide shafts 70 allow first member 20 and second member 30 to move linearly relative to each other (e.g., along axis 15) in the direction of arrows 91, 92. Friction reduction elements (e.g.: linear bushings or bearing) may be provided within shaft bores 26, 36 between members 20, 30 and guide shafts 70 to enable relatively smooth, consistent relative movement between members 20, 30.

Guide shafts 70 guide and control the direction of movement of first member 20 and second member 30. Specifically, guide shafts 70 permit the back-and-forth linear movement of first member 20 relative to second member 30 substantially parallel to axis 15, guide shafts 70, and the x-axis, and generally in the direction of arrows 91, 92. However, guide shafts 70 restrict the relative movement of first member 20 and second member 30 in y- and z-directions (i.e., in directions parallel to the y-axis and z-axis).

In the embodiment shown in FIGS. 1-3, two guide shafts 70 are provided between first member 20 and second member 30. However, in general, one or more guide shafts 70 may be provided to linearly couple first member 20 and second member 30. Although guide shafts 70 are provided in device 10 to guide the linear relative motion between members 20, 30, in general, any suitable mechanism may be employed to guide and restrict the relative motion of members 20, 30 including, without limitation a guidance frame, a track or rail system, or combinations thereof. For instance, in one embodiment, members 20, 30 are directly coupled and permitted to move linearly relative to each other, thereby eliminating the need for guide shafts 70.

The actuator 40 may be any suitable means or mechanism for providing an oscillatory motion to connecting rod 50. For example, the oscillator may comprise a motor, for example a battery powered motor, and a mechanical linkage between the motor and the connecting rod. The mechanical linkage may include a disk, a cam, a four bar linkage, etc.

Referring still to FIGS. 1-3, actuator 40 is fixed to first end 21 of first member 20 by mounting bracket 45 such that actuator 40 does not move translationally or rotationally relative to first member 20. In this embodiment, mounting bracket 45 is a separate component that is coupled to both first member 20 and actuator 40. However, in different embodiments, mounting bracket 45 is integral with first member 20. In addition, although actuator 40 is shown coupled to first end 21 of first member 20, in general, actuator 40 may be coupled to any suitable location of first member 20 including, without limitation, at second end 22 or at any location between ends 21, 22. Still further, although only a single actuator 40 is shown coupled to first member 20, actuator 40 may alternatively be coupled to second member 30 or coupled to both first member 20 and second member 30. In other embodiments, more than one actuator 40 is coupled to device 10. As will be explained in more detail below, actuator 40 is adapted to move second member 30 linearly relative to first member 20.

In some embodiments, the connecting rod 50 may be rigid, for example a metal, composite, plastic, or ceramic rod. When rigid, the connecting rod 50 may allow for both dynamic tension and compression of the fracture, as is described in more detail herein. In some embodiments, the connecting rod may be flexible, for example a rubber or elastomeric rod, band, strip, or the like. When flexible, the connecting rod 50 may allow for dynamic compression of the fracture. The following description details an embodiment having a rigid connecting rod 50, with the understanding that modifications could be made to accommodate use of a flexible connecting rod 50. For example, a flexible rod may be connected between the first member 20 and the second member 30 and dynamically tensioned at the first and/or second member.

First member 20 further comprises a rod coupling 24 extending from upper surface 27. Rod coupling 24 has a through bore 24a within which connecting rod 50 is slidingly disposed. Rod coupling 24 slidingly couples first member 20 to connecting rod 50, and further, guides the direction of sliding engagement of connecting rod 50 relative to first member 20. Specifically, rod coupling 24 permits the back-and-forth linear movement of connecting rod 50 relative to first member 20 substantially parallel to axis 15, guide shafts 70, and the x-axis, and in the direction of arrows 91, 92. Rod coupling 24 may include a friction reduction element (e.g., linear bushing or bearing) that enables relatively smooth, consistent sliding engagement of rod coupling 24 and connecting rod 50.

In this embodiment, rod coupling 24 also comprises a rod securing mechanisms 24b adapted to releasably fix connecting rod 50 to rod couplings 24 and first member 20. Specifically, rod securing mechanism 24b has a released position in which connecting rod 50 may be slid through bore 24a, and a fixed position in which connecting rod 50 is fixed to first member 20 (i.e., connecting rod 50 is not permitted to move translationally relative to first member 20). Rod securing mechanism 24b may comprise any suitable mechanism to releasably secure connecting rod 50 to first member 20 including, without limitation, a set screw, pins, clamp, or combinations thereof. In this exemplary embodiment, rod securing mechanism 24b comprise a set screw that is loosened to allow sliding engagement and adjustment of the linear position of connecting rod 50 relative to first member 20, and is tightened to secure and fix connecting rod 50 to first member 20.

Referring still to FIGS. 1-3, in this embodiment, rod coupling 24 is integral with first member 20 such that rod coupling 24 does not move rotationally or translationally relative to first member 20. In other embodiments, rod coupling 24 may comprise a separate part or component that is fixed to first member 20 by any suitable means including, without limitation, screws, bolts, pins, welding, or combinations thereof. Further, although rod coupling 24 is shown as extending from upper surface 27 of first member 20, in general, rod coupling 24 may be positioned at any suitable location of first member 20.

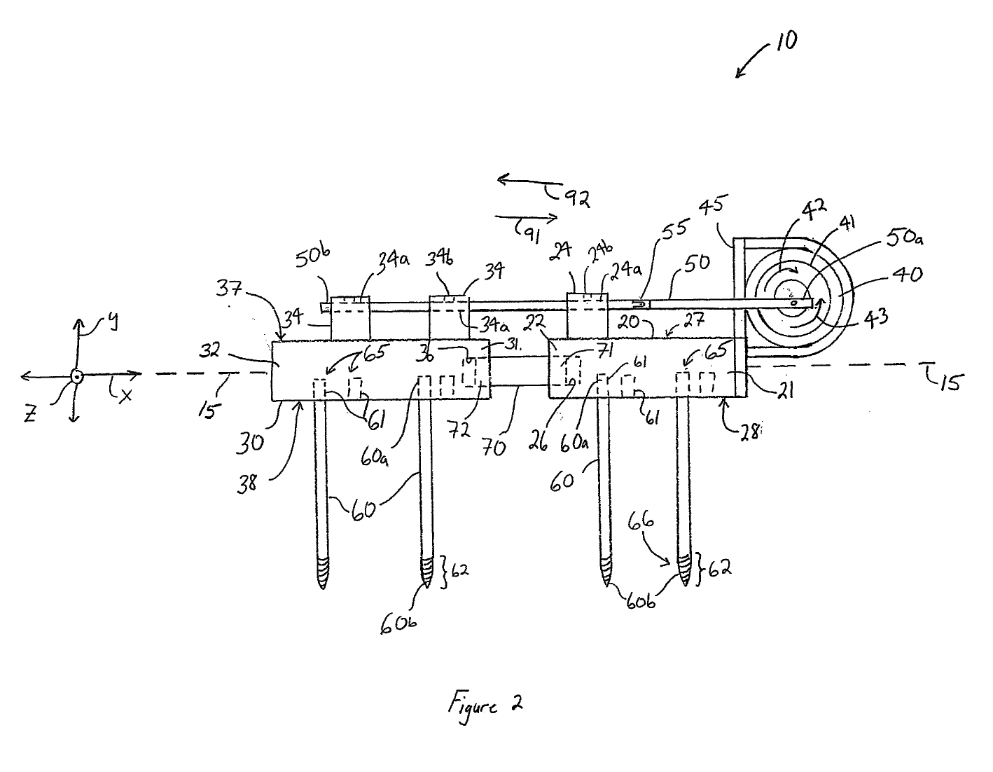

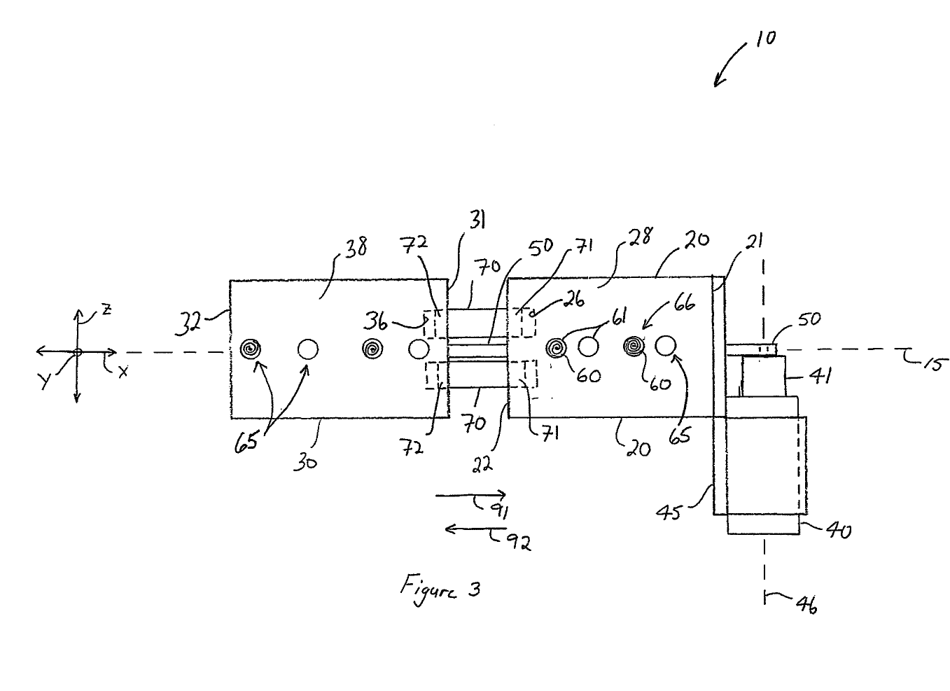

Referring specifically to FIGS. 2 and 3, two pins 60 extend from lower surface 28 of first member 20. Each pin 60 includes a fixed end 60a that is secured to first member 20 and a free end 60b distal first member 20 and device 10. As used herein, the term “distal” may be used to refer to components or positions that are relatively away or further from another component or position. Specifically, a pin coupling or connector 65 is provided to couple fixed end 60a of each pin 60 to first member 20. Pin connector 65 may comprise any suitable means or mechanism that couples fixed end 60a of pins to first member 20. Pins 60 are preferably releasably secured to first member 20 such that pins 60 do not move translationally or rotationally relative to first member 20 when secured to first member 20, but may also de-coupled or disengaged from first member 20 as desired.

In the exemplary embodiment shown in FIGS. 2 and 3, pin connector 65 comprises a mating socket 61 provided in lower surface 28 of first member 20. To secure pins 60 to first member 20, fixed end 60a is disposed and secured within a mating socket 61. Pins 60 may be secured within sockets 61 by any suitable means including, without limitation, mating threads, an adhesive, welding, a set screw or pin, or combinations thereof. It should be appreciated that other suitable mechanisms or means may be provided to couple pins 60 to first member 20 including, without limitation, mating slot and key coupling between each pin 60 and first member 20, a slideable rail system between pins 60 and first member 20, a quick release connection between pins 60 and first member 20, etc.

As will be explained in more detail below, during use of device 10, free ends 60b of each pin 60 are secured to the fractured bone of the patient. Thus, free end 60b of each pin 60 includes a bone coupling 66 adapted to couple pins 60 to the fractured bone of a patient. In the embodiment illustrated in FIGS. 1-3, bone coupling 66 on each pin 60 comprises threads 62 that are screwed into the fractured bone, thereby securing pins 60 to the bone. In this embodiment, adjacent sockets 61 are arranged in a straight line. However, in other embodiments, adjacent sockets 61 may be skewed or offset relative to one another. Since pins 60 connect device 10 to the fractured bone of the patient, pins 60 may also be referred to herein as “bone engagement pins.”

Referring still to FIGS. 2 and 3, although only two pins 60 are shown extending from first member 20, more than two pin connectors 65 are provided. Specifically, in this exemplary embodiment, four mating sockets 61 are provided in lower surface 28. By employing additional pin connectors 65 (e.g., sockets 61), the positioning of one or more pins 60 may be varied and/or more than two pins 60 may be secured to first member 20 as desired. In other words, by including a plurality of pin couplings (e.g., sockets 61), the versatility and adaptability of device 10 is enhanced.

Referring again to FIGS. 1-3, second member 30 includes a first end 31 proximal first member 20 and linearly coupled first member 20, and a second end 32 distal first member 20. As previously described, guide shafts 70 couple first end 31 of second member 30 to second end 22 of first member 20 such that second member 20 is free to move linearly relative to first member 20 in the direction of arrows 91, 92 (i.e., parallel to axis 15, guide shafts 70, and the x-axis). However, guide shafts 70 restrict relative movement of members 20, 30 in the y- and z-directions.

Second member 30 also includes two rod couplings 34, each comprising a through bore 34a and a rod securing mechanism 34b. Connecting rod 50 is disposed through each bore 34a. Similar to rod securing mechanism 24b previously described, rod securing mechanisms 34b are employed to releasably fix connecting rod 50 to rod couplings 34 and second member 30. Rod securing mechanism 34b may comprises any suitable mechanism to releasably secure connecting rod 50 to second member 30 including, without limitation, a set screw, pins, clamp, an interference fit, or combinations thereof. In this embodiment, rod securing mechanisms 34b each comprise a set screw that is loosened to allow sliding engagement and adjustment of the linear position of connecting rod 50 relative to second member 30, and is tightened to secure and fix connecting rod 50 to second member 30. Although the embodiments shown in FIGS. 1-3 show each rod coupling 34 as including a rod securing mechanism 34b, in other embodiments, one or more rod couplings 34 may include a rod securing mechanism 34b.

Rod couplings 24b, 34b permit members 20, 30, respectively, to be releasably fixed to connecting rod 50. When either rod securing mechanism 34b is in the fixed position, second member 30 is fixed to connecting rod 50. Likewise, when rod securing mechanism 24b is in the fixed position, first member 20 is fixed to connecting rod 50. Consequently, when either rod securing mechanism 34b is in the fixed position and rod securing mechanism 24a is also in the fixed position, connecting rod 50 is not free to move relative to first member 20 or second member 30 and the linear displacement between first member 20 and second member 30 is fixed. However, when either rod securing mechanism 34b is in the fixed position, and rod securing mechanism 24b is in the released position, connecting rod 50 is free to move linearly in the direction of arrows 91, 92 relative to first member 20, but does not move relative to second member 30. Lastly, when rod securing mechanism 24b is in the fixed position and both rod securing mechanisms 34b are in the released position, connecting rod 50 is fixed relative to first member 20, however second member 30 is free to move linearly relative to connecting rod 50 (i.e., connecting rod 50 slidingly engages bores 34a). In addition to, or as an alternative to rod securing mechanism 24b, a mechanism to releasably fix connecting rod 50 relative to first member 20 and/or second member 30 may be provided in guide shafts 70.

In the embodiment illustrated in FIGS. 1-3, rod couplings 24, 34 are integral with members 20, 30, respectively. In other embodiments, one or more rod coupling 24, 34 may comprise a separate part or component that is fixed to first member 20 or second member 30 by any suitable means including, without limitation, screws, bolts, pins, welding, or combinations thereof. Still further, in this embodiment, rod couplings 24, 34 extend from upper surface 27, 37 of members 20, 30, respectively, however, in general, each rod coupling 24, 34 may be positioned at any suitable location of first member 20 or second member 30, respectively, including without limitation on upper surfaces 27, 37, on lower surfaces 28, 38, along either side extending between upper surfaces 27, 37 and lower surfaces 28, 38, etc. It should be appreciated that rod couplings 24, 34 are substantially linearly aligned such that the substantially straight elongate connecting rod 50 may pass through each bore 24a, 34a simultaneously, without bending or breaking connecting rod 50. Thus, although rod couplings 24, 34 may be disposed in a variety of suitable positions, it is preferred that rod couplings 24, 34 are substantially linearly aligned.

Referring specifically to FIGS. 2 and 3, similar to first member 20, two pins 60 as previously described extend from lower surface 38 of second member 30. Each pin 60 includes a fixed end 60a secured to second member 30, and a free end 60b distal second member 30 and device 10. As previously described, a pin coupling or connector 65 is provided to couple fixed end 60a of each pin 60 to second member 30. Pin connector 65 may comprise any suitable means or mechanism that couples fixed end 60a of pins to second member 30. Pins 60 are preferably releasably secured to second member 30.

In the exemplary embodiment shown in FIGS. 2 and 3, pin connectors 65 comprise a mating socket 61 provided in lower surface 38 of second member 30. However, it should be appreciated that other suitable mechanisms or means may be provided to couple pins 60 to second member 30 including, without limitation, mating slot and key coupling between each pin 60 and second member 30, a slideable rail system between pins 60 and second member 30, a quick release connection between pins 60 and second member 30, etc.

Although pins 60 are positioned substantially perpendicular to lower surface 28, 38 of members 20, 30, respectively, in different embodiments, the configuration and orientation of one or more pin connectors 65 may permit one or more pin 60 to be oriented at an acute angle relative to lower surfaces 28, 38. For example, one or more mating socket 61 may be drilled into first member 20 at an acute angle relative to lower surface 28.

In the embodiments described herein, pins 60 are described as separate components that are coupled to first member 20. However, in different embodiments, pins 60 are formed integral with first member 20 and/or second member 30.

Referring again to FIGS. 1-3, connecting rod 50 is a substantially straight, elongate body including an actuator end 50a coupled to actuator 40 and a fixed end 50b that is releasably fixed to second member 30. Actuator end 50a may be coupled to actuator 40 by any suitable means including, without limitation, a pin, a ball-and-socket joint, etc. As will be explained in more detail below, the combination of actuator 40 and connecting rod 50 transform the rotary motion of actuator 40 into a linearly displacing motion of connecting rod 50 in directions substantially parallel to axis 15, guide shafts 70, and the x-axis in the direction of arrows 91, 92.

Connecting rod 50 is disposed through bores 24a, 34a and is linearly actuated by actuator 40. As previously described, rod couplings 24b, 34b permit members 20, 30, respectively, to be releasably fixed to connecting rod 50. When both first member 20 and second member 30 are fixed to connecting rod 50 (e.g., rod securing mechanism 34b and rod securing mechanism 24b are both in the fixed position), connecting rod 50 is not free to move relative to first member 20 or second member 30. In this configuration, the displacement of second member 30 relative to first member 20 is fixed as desired, and actuator 40 is restricted from inducing linear movement of second member 30 relative to first member 20. However, when second member 20 is fixed to connecting rod 50 (e.g., either rod securing mechanism 34b is in the fixed position) and first member slidingly engages connecting rod 50 (e.g., rod securing mechanism 24b is in the released position), connecting rod 50 is free to move linearly in the direction of arrows 91, 92 relative to first member 20, but does not move relative to second member 30. In this configuration, actuator 40 is permitted to linearly move connecting rod 50 and second member 30 in the direction of arrows 91, 92 relative to first member 20. Lastly, when first member 20 is fixed to connecting rod 50 (e.g., rod securing mechanism 24b is in the fixed position), and both rod securing mechanisms 34b are in the released position, connecting rod 50 is restricted from moving relative to first member 20, however, second member 30 is free to move linearly relative to connecting rod 50 (i.e., connecting rod 50 slidingly engages bores 34a). In this configuration, actuator 40 is restricted from moving second member 30 relative to first member 20, even though second member 30 may move linearly relative to first member 20.

When actuator 40 linearly displaces connecting rod 50, connecting rod 50 moves relative to first member 20 without displacing first member 20, however, connecting rod 50 does not move relative to second member 30 and therefore linearly displaces second member 30 relative to first member 20. Thus, the displacement of second member 30 relative to first member 20 is initiated and controlled by actuator 40 via connecting rod 50, and is guided rod couplings 24, 34 and guide shafts 70.

Referring to FIGS. 1 and 2, in this embodiment, connecting rod 50 also comprises a pivot joint 55 along its length generally between actuator end 50a and rod coupling 24 of first member 20. Joint 55 permits slight displacement of actuator end 50a in the y-direction without displacing first member 20 or second member 30 in the y-direction. For instance, in this embodiment, actuator end 50a is moved rotationally in a direction of arrow 42 or arrow 43 by actuator 40. This rotational movement of actuator end 50a is converted to the linear movement of connecting rod 50 and second member 30 relative to first member 20. As actuator end 50a is rotationally displaced in the x-y plane, actuator end 50a will experience displacement in the x-direction and displacement in the y-direction. Joint 55 permits the displacement of actuator end 50a in the y-direction without transmitting this displacement to the remainder of connecting member 50. However, it is to be understood that joint 55 does transmit forces and displacement in the x-direction. In alternative embodiments where actuator end 50a does not undergo displacement in the y-direction, joint 55 may not be necessary. Such an embodiment may include a cam shaft mechanism.

Although axis 46 of disc 41 is illustrated as substantially parallel to upper surface 27 in FIGS. 1-3, it should be appreciated that disc 41 may alternatively be oriented with axis 46 at any suitable angle relative to upper surface 27. For instance, in one exemplary embodiment, disc 41 is oriented on its side such that axis 46 is substantially perpendicular to upper surface 27.

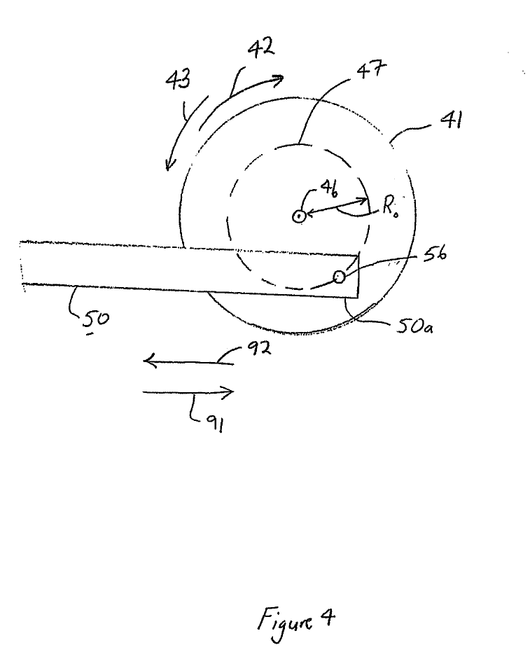

Referring now to FIGS. 1 and 4, as previously described, actuator 40 is fixed to first member 20 and is coupled to actuator end 50a of connecting rod 50. In general, actuator 40 induces controlled linear displacement of connecting rod 50 and second member 20 relative to first member 20. In general, actuator 40 may comprise any suitable device for providing linear actuation or displacement to connecting rod 50 including or a flexible element replacing the connecting rod 50, without limitation, an electric motor, a hydraulic actuator, a pneumatic actuator, a piezo-electric actuator, an electromagnetic actuator, or the like. In this embodiment, actuator 40 comprises an electric motor that rotates a disc or actuation member 41. In an exemplary embodiment, actuator 40 is a 15.6 V DC electric motor. In embodiments where actuator 41 is an electric motor or electrical device, power may be provided by any suitable means including, without limitation, batteries, a wall outlet, or combinations thereof. Disc 41 has a central axis 46 and may be rotated about axis 46 in the direction of arrow 42 or arrow 43.

As best shown in FIG. 4, actuator end 50a of connecting rod 50 is coupled to disc 41. In particular, actuator end 50a is coupled to disc 41 radially offset from axis 46 by a radial offset distance Rₒ. As disc 41 rotates about axis 46, actuator end 50a of connecting rod 50 rotates through a circular path 47 of radius Rₒ. As actuator end 50a rotates about circular path 47 it oscillates in the y-direction by a distance or amplitude Rₒ relative to axis 46, and oscillates in the x-direction by a distance or amplitude Rₒ relative to axis 46.

By controlling the rotation of disc 41 with actuator 40 and the radial offset Rₒ of actuator end 50a, the movement and/or displacement of second member 30 relative to first member 20 may be varied and controlled. For oscillatory motion of second member 30 relative to first member 20, disc 41 is rotated, thereby causing actuator end 50a, and hence second member 30, to oscillate in the x-direction (i.e., in the direction of arrows 91, 92) by a distance or amplitude Rₒ. It is to be understood that oscillations having an amplitude Rₒ result in a maximum displacement of second member 30 relative to first member 20 by a distance 2*Rₒ. Alternatively, for a fixed displacement of second member 30 relative to first member 20, disc 41 may be rotated until actuator end 50a, and hence second member 30, is positioned at the desired displacement from first member 20. Once the desired displacement is achieved, rotation of disc 41 may be stopped, thereby locking in the displacement of second member 30 relative to first member 20.

In the manner described, second member 30 may be linearly oscillated by a desired amplitude and/or linearly displaced by a desired distance relative to first member 20. The displacement of second member 30 relative to first member 20 may vary with time (i.e., rotate disc 41) or the displacement of second member 30 relative to first member 20 maintained or fixed as desired (i.e., no rotation of disc 41). By varying the radial offset Rₒ of actuator end 50a relative to axis 46, the range of motion and displacement of second member 30 relative to first member 20 may be varied as desired. For instance, if radial offset Rₒ is increased, the potential linear displacement of second member 30 relative to first member 20 is increased. To the contrary, if radial offset Rₒ is decreased, the potential linear displacement of second member 30 relative to first member 20 is decreased. It should be noted that if the connecting rod 50 is replaced by a flexible member (e.g. rubber element) the oscillatory motion amplitude is an indicator of the relative force magnitude compared to travel distance explained in detail above. However, the same principal still applies.

It should be appreciated that by varying the power and speed of actuator 40 (e.g., rotational speed of actuator 40), the forces and travel distance applied to second member 30 via connecting rod 50, and the frequency of oscillation of second member 30 relative to first member 20 may be varied and controlled. For instance, in embodiments where actuator 40 is an electric motor, the frequency of oscillation of second member 30 may be varied by adjusting the voltage and current of the electric motor. Thus, in embodiments in which actuator 40 is an electric motor, a voltage or current regulator (e.g. potentiometer with variable resistance) may be electrically coupled to the electric motor to allow the user to alter the power and frequency, and hence the performance, of the electric motor.

Although actuator end 50a is shown directly connected to disc 41 of actuator 40, in other embodiments, one or more additional components (e.g., ball bearing, etc.) may be provide between actuator end 50a and actuator 40.

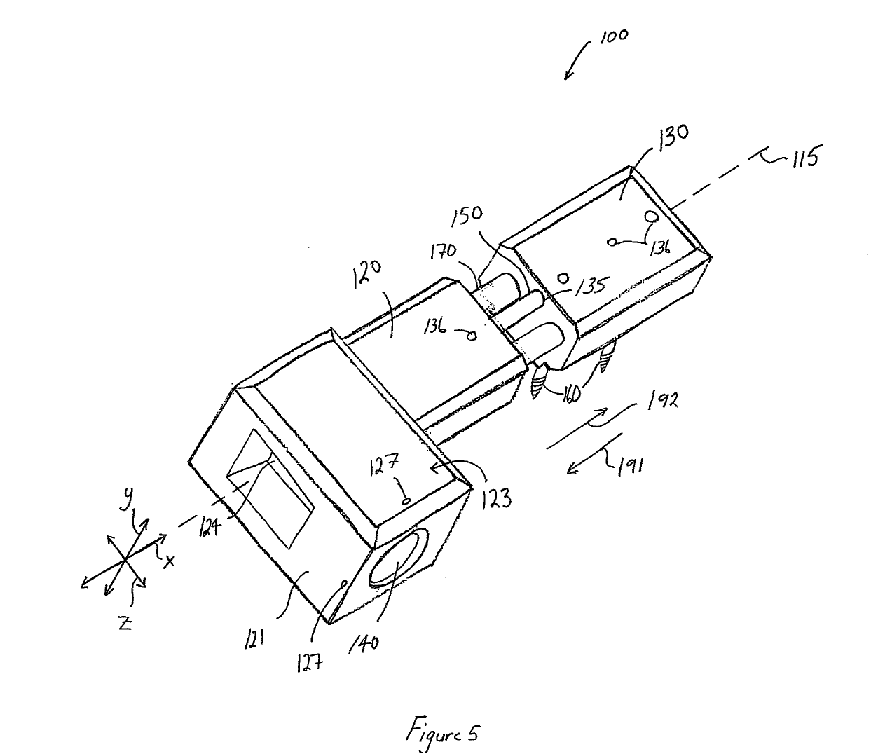

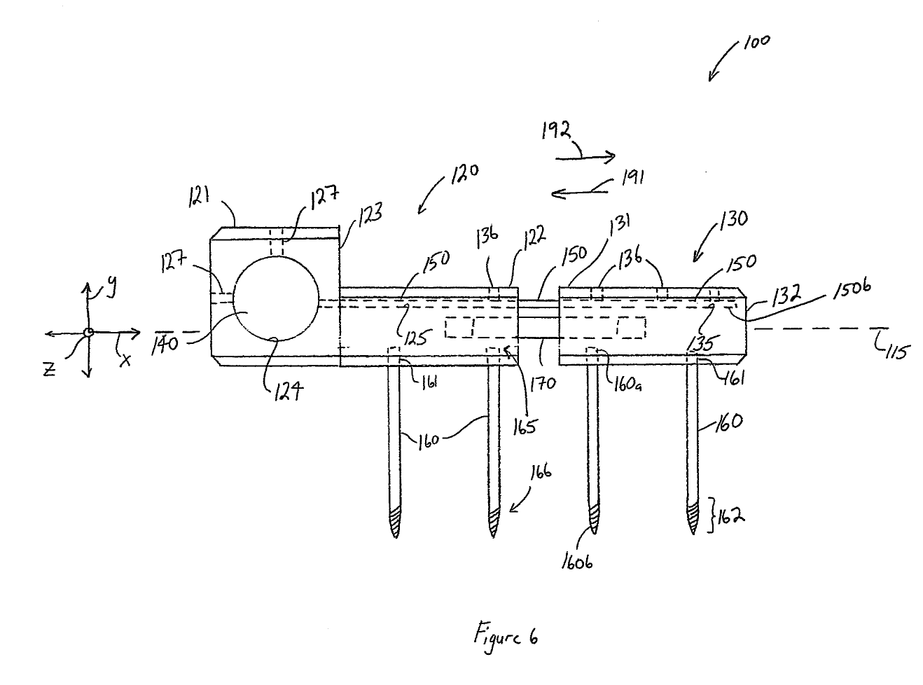

Referring now to FIGS. 5 and 6, another embodiment of a bone fixation and dynamization device 100 having a longitudinal axis 115 is illustrated. Bone fixation and dynamization device 100 comprises a first member 120, a second member 130, an actuator 140, pins 160, and a connecting rod 150. First member 120 has a first end 121 that includes an integral housing 123 and a second end 122 linearly coupled to second member 130. Housing 123 includes an inner cavity 124 that accommodates actuator 140. Specifically, actuator 140 is disposed within cavity 124 and coupled to housing 123. In this embodiment, actuator 140 is coupled to housing 123 by set screws 127 shown in FIGS. 5 and 6.

Similar to device 10 previously described, first member 120 is linearly moveable relative to second member 130 in the direction of arrows 191, 192. Namely, a pair of guide shafts 170 between first member 120 and second member 130 guide the movement of first member 120 relative to second member 130. Guide members 170 permit linear movement of first member 120 relative to second member 130 in the x-direction (e.g., parallel to axis 115), but restrict relative movement in the y- and z-directions. First member 120 and second member 130 each include a rod bore 125, 135, respectively, within which connecting rod 150 is disposed. As desired, rod securing mechanism(s) 136 (e.g., set screws) may be used to fix first member 120 and/or second member 130 to connecting rod 150.

Connecting rod 150 has an actuator end (now shown) coupled to actuator 140 and a free end 150b that is coupled to second member 130. Actuator 140 is adapted to linearly displace connecting rod 150 and hence, linearly displace second member 130 relative to first member 120 in the direction of arrows 191, 192.

Two pins 160 extend from first member 120 and two pins 160 extend from second member 130. Each pin 160 includes a fixed end 160a coupled to first member 120 or second member 130, and a free end 160b distal device 10. Pins 160 are coupled to members 120, 130 by pin connectors 165. In this embodiment, within a mating socket 161 provided in member 120, 130. In this exemplary embodiment, pin connectors 165 comprise mating sockets 161 within which fixed ends 160a are releasably disposed and secured. Free end 160b of each pin 160 includes a bone coupling 166 adapted to secure pins 160 to the fractured bone of a patient. In this embodiment, bone couplings 166 each comprise threads 162.

Bone fixation and dynamization device 100 operates substantially the same as device 10 previously described. Actuator 140 controls the displacement of second member 130 relative to first member 120, and further, the relative motion and displacement between first member 120 and second member 130 may be varied by controlling actuator 140.

As compared to device 10 previously described, device 100 includes several unique features. For instance, device 100 employs a simplified design in which first member 120 includes an integral housing 123. Integral housing 123 reduces the need for an external coupling frame or bracket to secure actuator 140 to fist member 120, shields the moving actuator 140 from the patient, and reduces the number of mechanical connections in device 100 that may loosen over time due to vibrations. As another example, device 100 utilizes internal bores 125, 135 to accommodate connecting rod 150. Inner bores 125, 135 eliminate the need for external rod couplings (e.g., rod couplings 24, 34) and associated mechanical connections, and substantially shields the moving connecting rod 150 from patient.

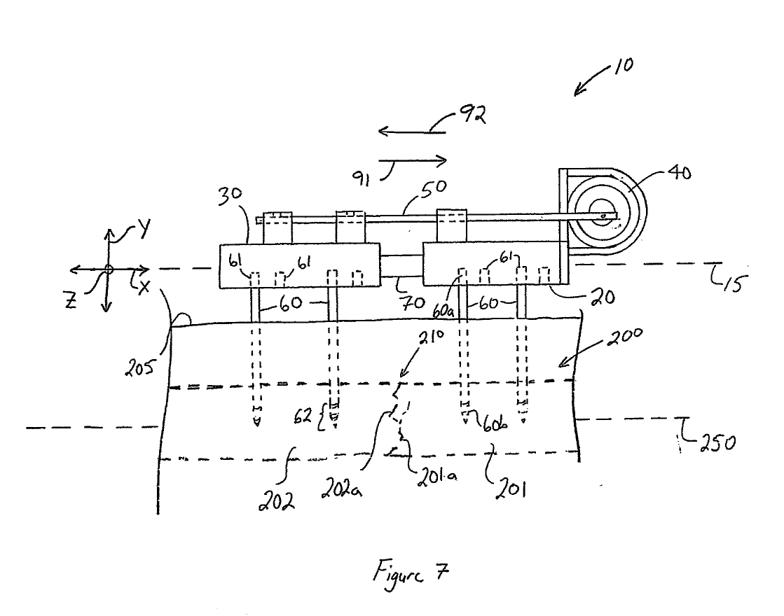

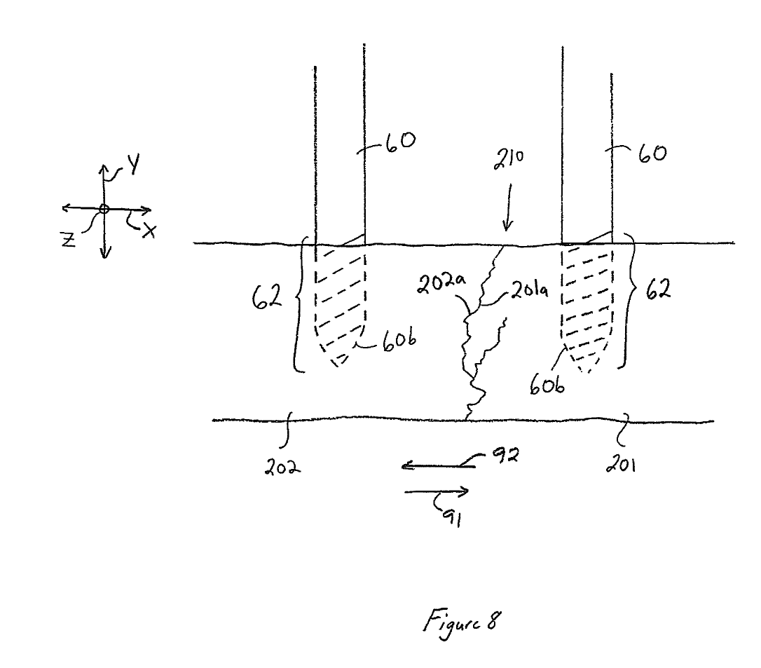

Referring now to FIGS. 7 and 8, bone fixation and dynamization device 10 is coupled to a bone 200 having a longitudinal axis 250. Bone 200 includes a fracture or cut 210 along its length, resulting in two bone segments 201, 202, one on either side of fracture 210. Fracture or cut 210 may be caused by an accident (fracture) or by a surgically induced osteotomy (cut). In cases where fracture 210 is a surgically induced osteotomy, it may be referred to as a “cut”. For purposes of the discussion to follow, fracture or cut 210 will be termed a “fracture”, it being understood that distraction osteogenesis and other types of surgically induced bone fragmentations may be treated similarly included. Each fracture segment 201, 202 includes a fracture end 201a, 202a, respectively, that generally opposes each other.

Device 10 is percutaneously coupled to bone 200 via pins 60 with first member 20 percutaneously coupled to fracture segment 201 and second member 30 is percutaneously coupled to fracture segment 202. In other words, first member 20 is coupled to bone 200 on one side of fracture 210 and second member 30 is coupled to bone 200 on the opposite side of fracture 210. Each pin 60 is secured to bone 200 by inserting and screwing threads 62 of free ends 60b into bone 200. Thus, pins 60 may also be referred to herein as “bone engagement pins.” The positioning of the pins 60 inside the bone may include unicortical or bicortical impingement.

Device 10 is positioned external to the patient, with lower surfaces 28, 38 facing the patient, and bone engagement pins 60 passing through the patients skin and underlying tissues to bone 200, thereby coupling device 10 to bone 200. Since, lower surfaces 28, 38 face the patient when device 10 is coupled to the patient, lower surfaces 28, 38 may also be referred to herein as “patient facing surfaces.”

In some embodiments, pins 60 are secured to bone 200 prior to coupling members 20, 30 to pins 60. For instance, each pin 60 may be independently fixed to bone 200 with threads 62. Then, after free end 60b of each pin 60 is properly secured to bone 200, fixed ends 60a of each pin is secured to first member 20 or second member 30 via pin connectors 65 (e.g., set screws, clamps, etc.). In such an example, pins 60 are preferably sufficiently aligned and spaced when secured to bone 200 such that they will be substantially aligned with mating sockets 61 when members 20, 30 are coupled to pins 60. In some embodiments, first member 20 and second member 30 are made of multiple components coupled together. This allows positioning of the pins 60 based on surgical preference instead of alignment of the device. Still further, in other embodiments, pins 60 are secured to bone 200 while secured to members 20, 30. For instance, access holes (not shown) through members 20, 30 or extension of pins 60 through upper surfaces 27, 37 (not shown) may permit manipulation of pins 60 while pins 60 are coupled to members 20, 30 (e.g., pins 60 may be screwed into bone 200 while coupled to members 20, 30).

In the embodiment shown in FIG. 7, four pins 60 are secured to bone 200, two pins 60 on either side of fracture 210. However, in other embodiments, one or more pin 60 may be secured to bone 210 on either side of fracture 210. Further, the spacing of pins 60 may be adjusted by selecting which mating sockets 61 each pin 60 is disposed within.

Once pins 60 are secured to fracture segments 201, 202 and secured to members 20, 30, device 10 stabilizes and immobilizes fracture segments 201, 202 and fracture 210. Proper stabilization and immobilization of fracture segments 201, 202 and fracture 210 places fracture ends 201a, 202a in contact and allows a callus of tissue to form and harden around fracture 210 during normal fracture healing. Specifically, the axial positions and radial positions of fracture segments 201, 202 may be controlled via device 10. As used herein, the terms “axial” and “axially” refer to positions or movement generally along a central axis (e.g., axis 250), whereas the terms “radial” or “radially” refer to positions or movement generally perpendicular to a central axis (e.g., axis 250). For instance, the radial positions of fracture segments 201, 202 may be adjusted relative to each other by adjusting the relative depth of each pin 60 in fracture segments 201, 202. In addition, the linear displacement of second member 30 relative to first member 20 results in substantially the same linear displacement of fracture segment 202 relative to fracture segment 201.

Further, once pins 60 are secured to bone segments 201, 202, depending on the linear position of second member 30 relative to first member 20, fractured bone 200 may be placed in tension by pushing segments 201, 202 apart or compression by pushing segments 201, 202 together. For instance, when second member 30 is urged in the direction of arrow 92 relative to first member 20 (i.e., actuator 40 is pushing members 20, 30 apart), bone 200 will be placed in tension and bone segments 201, 202 will be pushed apart at fracture 210. However, when second member is urged in the direction of arrow 91 (i.e., actuator 40 is pushing members 20, 30 together), bone 200 will be placed in compression and bone segments 201, 202 will be pushed together at fracture 210. Note that for embodiments where the connecting rod 50 is a flexible element, typically compression forces are applied to the bone segments.

As previously described, device 10 may be employed to stabilize and immobilize bone segments 201, 202 and fracture 210, and/or to place bone 200 in tension or compression. By stabilizing bone segments 201, 202 and controllably placing bone 200 in tension, device 10 may be used to lengthen bone 200 via distraction osteogenesis. Distraction osteogenesis is a technique generally used by orthopedic surgeons to lengthen bones and hence limbs. For instance, if a patient has one leg that is slightly shorter than the other, distraction osteogensis may be employed to lengthen the shorter leg to match the lengths of both legs. Distraction osteogenesis typcially involves urging the bone segments of a fractured bone apart as the callus tissue forms therebetween. However, before the callus tissue mineralizes and hardens, the bone segments are further urged apart, and callus tissue is again allowed to form therebetween. This process is repeated until the desired bone length is achieved, at which time the callus tissue between the bone segments is allowed to mineralize and harden. Thus, by pulling the bone segments apart stepwise and before the callus tissue fully mineralize and harden into bone, the surgeon can effectively lengthen a bone and limb.

Referring still to FIGS. 7 and 8, by controllably placing bone 200 in tension by urging bone segment 201, 202 apart at fracture 210, device 10 offers the potential to lengthen bone 200 via distraction osteogenesis. For instance, bone segments 201, 202 are slightly and controllably urged apart by device 10 as previously described and callus tissue is allowed to begin forming between bone segments 201, 202 at fracture 210. However, before the callus tissue mineralizes or hardens, bone segments 201, 202 may be further urged apart, and additional callus tissue permitted to form therebetween. This process may be repeated until the desired bone length is achieved. Once the desired bone length is achieved, bone segments 201, 202 are stabilized and maintained in position by device 10 while the callus tissue formed therebetween is allowed to mineralize and harden. Once the callus tissue has hardened and fracture 210 has sufficiently healed, device 10 may be removed from a lengthened bone 200. In some embodiments, connecting rod 50 may include a gauge or scale to indicate the lengthening achieved through this process.

In addition, once pins 60 are secured to fracture segments 201, 202 and secured to members 20, 30, device 10 provides dynamization at fracture 210. Active dynamization may be employed to enhance healing of a normal bone fracture 210, or to enhance healing during the successive stages of distraction osteogenesis. Specifically, as second member 30 is oscillated relative to first member 20 as previously described, oscillations are induced at fracture ends 201a, 202a. It should be appreciated that as second member 30 is oscillated relative to first member 20, bone segments 201, 202 are oscillated between tension and compression. In other words, fracture ends 201a, 202a are compressed together, then pulled apart, then compressed together, and so on. The amplitude or distance of the oscillations, the frequency of the oscillations, the duration of the oscillations, and the loads induced by the oscillation are controlled by the actuator 40 and the radial offset Rₒ. The amplitude, frequency, and duration of oscillations, as well as the loads induced by the oscillations, are preferably optimized to enhance bone healing.

Thus, device 10 can fix the displacement of fracture segments 201, 202 relative to each other, or actively induce the micromovement of fracture segments 201, 202 relative to each other at fracture 210. These micromovements result in dynamization, which offers the potential to stimulate, promote, and accelerate and the healing of fracture 210. As desired, the amplitude of the oscillations, the duration of the oscillations, the frequency of the oscillations, and the forces induced by the oscillations may be adjusted depending on the application, patient comfort, and/or to compensate for changes in tissue and/or bone properties during healing.

As previously discussed, studies have shown that micromovements on the order of 1 mm or less enhance bone fracture healing. Thus, the amplitude of the oscillations are preferably less than 1 mm, and more preferably less than 0.5 mm. Such amplitudes are achieved in an exemplary embodiment in which actuator end 50a of connecting rod 50 is coupled to disc 41 with a radial offset Rₒ of about 0.5 mm. The 0.5 mm offset offers the potential for a maximum displacement of first member 20 relative to second member 30 of about 1 mm.

In addition, as previously discussed, studies have shown that oscillating micromovements having frequencies between 0.25 and 0.75 Hz, and more preferably about 0.5 Hz, enhance bone fracture healing. Thus, the frequency of the oscillating micromovements are preferably about 0.5 Hz. The frequency of the oscillating micromovements may be varied as desired by controlling actuator 40 as previously described. Therefore, in a preferred embodiment, bone fixation and dynamization device 10 applies oscillations to fracture 210 and segments 201, 202 having an amplitude of 1 mm or less and a frequency of about 0.5 Hz.

It should be understood that additional research and studies in the field of active bone dynamization may reveal additional and/or alternative preferred amplitudes and/or frequencies of oscillation. For instance, in one alternative embodiment, one may chose to work around the resonance frequency of the tissue and adjust the power and frequency based on the healing phase of the tissue. These preferred amplitudes and frequencies may be achieved by adjusting or changing out actuator 40 as necessary.

As described above, most conventional bone dynamization devices and techniques rely on the normal physical activities of the patient to load the fractured bone(s) in order to promote bone healing. Such conventional dynamization techniques may be insufficient for patients who are unable or unwilling to load their bones by physical activity, and insufficient for fractured bones that experience minimal or no loads during normal physical activities of the patient. In addition, such conventional dynamization techniques may be insufficient to promote bone healing in low gravity or micro-gravity environments in which physical activities do not result in sufficient loading of the bones. However, by actively inducing dynamization, embodiments of the bone fixation and dynamizer described herein (e.g., device 10, 100) offer the potential to provide sufficient dynamization to fractured bones without relying on the patient's physical activities to load and induce micromovements at the bone fracture site. Thus, embodiments described herein may be used with elderly, traumatized, paraplegics, or other individuals who are unable or otherwise unwilling to load their bones through normal activities.

In addition to load-bearing bones, embodiments described herein may also be used to provide sufficient dynamization to bones that typically do not experience adequate physiological loads through the normal activities of the patient. Further, since load bearing activities are not required to induce dynamization, embodiments described herein may be used in low gravity, microgravity, or zero gravity environments where there is minimal or no loading of bones. For example, device 10 may be applied on Earth or in microgravity environments (e.g., in space) to promote bone fracture healing. Thus, embodiments of the bone fixation and dynamization device disclosed herein offer the potential to overcome various problems of prior devices.

In the manner described, embodiments described herein provide devices and methods that offer the potential to immobilize a bone fracture, stabilize a bone fracture, lengthen a bone via distraction osteogenesis, promote bone healing, accelerate bone fracture healing, or combinations thereof. Enhancement of bone fracture healing may be achieved by the application of micromovements at the fracture site (e.g., active dynamization). Additional enhancement of bone fracture healing may also be achieved by the addition of vibration, ultrasound, or electromagnetic field therapy in other embodiments. The embodiments described herein offer potential benefits for patients unable to load their bones, for patients with fractures in bones that do not undergo loading, and in low gravity or micro-gravity environments. It should be understood that embodiments described herein may also be used to stabilize a fracture, lengthen a bone via distraction osteogenesis, and/or actively dynamize a bone fracture in normal and otherwise healthy patients and with bones that experience sufficient loading during normal physical activities.

The components of the bone fixation and dynamization devices disclosed herein (e.g., first member 20, 120, second member 30, 130, connecting rod 50, 150, guide shafts 70, 170, pins 60, 160, etc.) may comprise any suitable material including without limitation metals or metal alloys (e.g., aluminum, stainless steel, titanium, etc.), or non-metals (e.g., plastic, composite, etc.). To reduce the weight and bulkiness of the device, the first member (e.g., first member 20, 120) and the second member (e.g., second member 30, 130) preferably comprise a relatively lightweight, durable material such as a polymer (e.g., plastic) or composite (e.g., plaster reinforced with cyanoacrylate). In addition, the guide shafts (e.g., guide shafts 70, 170), and the pins (e.g., pins 60, 160) preferably comprises a relatively rigid, strong material capable of transmitting forces such as stainless steel, aluminum, titanium, or alloys formed therefrom. Furthermore, connecting rod (e.g., connecting rod 50, 150) preferably comprises a relatively rigid, strong material capable of transmitting forces such as stainless steel, aluminum, titanium, or alloys formed therefrom, However, in embodiments in which connecting rod 50 is a flexible member, it may be made of a rubber-like material and/or silicone (e.g., an elastomeric or rubber band). Since the pins pass through the skin, the underlying tissue of the patent, and are secured to the fractured bone, the pins preferably comprises a biocompatible material. The components of the bone fixation and dynamization devices disclosed herein may be formed by any suitable method including without limitation machining, molding, casting, or combinations thereof.

In certain embodiments, the bone fixation and dynamization devices disclosed herein may include sensors, diagnostic components, or other suitable means to monitor the healing of the bone fracture during treatment. In one exemplary embodiment, the power consumption (voltage (V) and current (I)) used by the actuator (e.g., actuator 40) of the bone fixation and dynamization device (e.g., device 10) is measured real time to monitor the fracture healing process. Specifically, the measured voltage (V) and current (I) of the actuator is used to calculate the actuator power consumption (P), where P=I*V. The power consumed by the actuator is correlated to the resistance to deformation of the fracture site, which in turn is an approximation of the tissue stiffness filling the fracture gap. In general, the power consumed by the actuator is directly related to the stiffness of the fracture site (e.g., as the stiffness of the fracture site increases, the power required to induce dynamization increases). Since the stiffness of the fracture site increases with time as the tissue at the fracture site heals and the callus tissue hardens, by monitoring the voltage (V) and current (I) of the actuator, it is possible to monitor the healing process.

A force sensor may be coupled to the controller and used to measure the forces (e.g., tensile and/or compressive) applied to the fracture. In an embodiment, the control feedback mechanism may comprise a force sensor, for example sensing the power consumption of the actuator, as described above. In an embodiment, the control feedback mechanism may comprise an alternative force sensor in addition to or in lieu of sensing the power consumption of the actuator. In such embodiments, where a rigid connecting rod 50 is used, the device may be displacement controlled, for example the radius Rₒ on the actuator disc determines that displacement may be applied at the fracture gap. In such embodiments, where a flexible connecting rod 50 (e.g., elastomeric or rubber band) is used, the device may be force controlled, for example the radius Rₒ on the actuator disc determines how much dynamic compressive force may be applied at the fracture gap.

In addition, in some embodiments, a closed-loop or open-loop control feedback mechanism is employed to adjust the amplitude and frequency of micromovements based on the monitored healing of the bone fracture. In an exemplary embodiment, a feedback signal, e.g., the actuator power consumption and/or the resonance frequency of the system is monitored as previously described. Thus, the feedback signal may be used to indicate the fracture site stiffness, and thus the healing phase of the patient. This information may be provided to a feedback mechanism that automatically adjusts the frequency and amplitude of the oscillating micromovements (e.g., by adjusting the voltage and current to the actuator and the radial offset Rₒ) as necessary to enhance bone healing rates. Alternatively, this information may be provided to a health care provider to allow the health care provider to adjust the frequency and amplitude of the oscillating micromovements as desired to enhance bone healing rates. As a result, an estimate may be made as to whether the patient will heal regularly and/or if treatment needs to continue or be adjusted.

In addition, it should be understood that embodiments of the bone fixation and dynamization device described herein (e.g., device 10, 100) may be used with various bones and various fracture types. To accommodate different sized bones, the dimensions of the device may be altered to create smaller scale or larger scale versions of the device that are applied as external fixation devices or even implanted.

While preferred embodiments have been shown and described, modifications thereof can be made by one skilled in the art without departing from the scope or teachings herein. The embodiments described herein are exemplary only and are not limiting. Many variations and modifications of the system and apparatus are possible and are within the scope of the invention. For example, the relative dimensions of various parts, the materials from which the various parts are made, and other parameters can be varied. In addition, it should be appreciated that the various parts may be reconfigured and still achieve the same functions. Accordingly, the scope of protection is not limited to the embodiments described herein, but is only limited by the claims that follow, the scope of which shall include all equivalents of the subject matter of the claims.

What is claimed is:

1. A bone fixation and dynamization device comprising:
a first member having a first end and a second end;
a second member having a first end and a second end, wherein the first end of the second member is coupled to the second end of the first member body, wherein the first member is linearly moveable relative to the second member;
an actuator coupled to the first member;
a feedback controller coupled to the actuator;
an elongate rod having an actuator end coupled to the actuator and a fixed end fixed to the second member, wherein the actuator is operable to move the rod and the second member linearly relative to the first member responsive to the feedback controller;
at least one bone engagement pin extending from the first member; and
at least one bone engagement pin extending from the second member.

2. The device of claim 1 wherein the first member comprises a through bore within which the rod is slidingly disposed.

3. The device of claim 2 wherein the second member comprises a through bore within which the fixed end of the rod is disposed.

4. The device of claim 1 wherein the first member comprises an actuator housing.

5. The device of claim 4 wherein the actuator housing is an integral housing having an inner cavity, wherein the actuator is disposed within the inner cavity and coupled to the housing.

6. The device of claim 1 wherein the first member and the second member each comprise at least one pin connector, wherein the at least one bone engagement pin extending from the first member includes a fixed end coupled to the pin connector of the first member and a distal end having threads adapted to fix the first member to a first bone segment, and wherein the at least one bone engagement pin extending from the second member includes a fixed end coupled to the pin connector of the second member and a distal end having threads adapted to fix the second member to a second bone segment.

7. The device of claim 1 further comprising at least one guide shaft that couples the second end of the first member to the first end of the second member, wherein the at least one guide shaft guides the linear movement of the first member relative to the second member.

8. The device of claim 7 further comprising two parallel guide shafts that couple the second end of the first member to the first end of the second member, wherein the at least one guide shaft guides the linear movement of the first member relative to the second member.

9. The device of claim 7, wherein the at least one guide shaft has a first member end disposed within a mating shaft bore in the second end of the first member and a second member end disposed within a mating shaft bore in the first end of the second member.

10. The device of claim 1 wherein the actuator comprises a disc having a central axis, wherein the actuator rotates the disc about the axis.

11. The device of claim 10 wherein the actuator end of the rod is radially offset a distance Rₒ from the axis of the disc.

12. The device of claim 11 wherein the radial offset distance Rₒ is less than or equal to 1 mm.

13. The device of claim 1, wherein said first member and the second member comprise a composite material.

14. The device of claim 1, wherein the actuator comprises an electric motor.

15. The device of claim 14 further comprising a power source and a voltage regulator electrically coupled to the electric motor, wherein the potentiometer is operable to adjust the speed and power of the electric motor.

16. The device of claim 14, wherein the power source comprises at least one battery.

17. The device of claim 14 further comprising a monitoring component to measure the power consumed by the electric motor.

18. A method for fixing and dynamizing a fracture in a bone, comprising:
a) providing a bone fixation and dynamization device, wherein the bone fixation and dynamization device comprises:
a first member;
a second member coupled to the first member, wherein the second member is operable to move linearly relative to the first member;
an actuator coupled to the first member;
a feedback controller coupled to the actuator; and
an elongate rod having an actuator end coupled to the actuator and a fixed end fixed to the second member, wherein the actuator is operable to move the second member linearly relative to the first member responsive to the feedback controller;
b) connecting the first member to a first bone segment on one side of the fracture;
c) connecting the second member to a second bone segment on the other opposite side of the fracture; and
d) applying oscillating micromovements to the first and second bone segments with the bone fixation and dynamization device.

19. The method of claim 18 wherein the first member comprises at least one bone engagement pin percutaneously connected to the first bone segment, and the second member comprises at least one bone engagement pin percutaneously connected to the second bone segment by the at least one bone engagement pin.

20. The method of claim 19 wherein the first member comprises two bone engagement pins percutaneously connected to the first bone segment and the second member comprises two bone engagement pins percutaneously connected to the second bone segment.

21. The method of claim 18 wherein the oscillatory micromovements have an amplitude of less than or equal to 1 mm.

22. A method of dynamizing a fracture in a bone having a longitudinal axis comprising:
engaging a bone segment on each side of the fracture with at least one bone engagement pin;
oscillating the bone engagement pins on either side of the fracture linearly relative to one another;
applying linear oscillating micromovements the bone segments on either side of the fracture; and
controlling the micromovements via feedback control.

23. The method of claim 22 wherein the bone engagement pins are percutaneously coupled to the bone segments on either side of the fracture.

24. The method of claim 22 wherein the linear micromovements applied to the bone segments are less than or equal to 1 mm.

25. The method of claim 22 wherein the bone engagement pins are oscillated by an elongate rod coupled to the bone engagement pins.

26. The method of claim 22 wherein the bone engagement pins are oscillated in compression by a flexible band coupled to the bone engagement pins.

27. The method of claim 22 wherein the linear oscillating micromovements comprise the application of compressive forces, tensile forces, or both to the fracture.
